(19) United States
(12) Patent Application Publication
(10) Pub. No.: US 2026/0240445 A1
(43) Pub. Date: Aug. 20, 2026

(54) METHOD AND DEVICE FOR ESTIMATING VO2 OF USER USING WIRELESS SIGNAL AROUND USER

(71) Applicant: KOREA INSTITUTE OF SCIENCE AND TECHNOLOGY, Seoul (KR)

(72) Inventors: Heedong KO, Seoul (KR); Byounghyun YOO, Seoul (KR); Muhammad ATIF, Seoul (KR); Bumho LEE, Seoul (KR)

(73) Assignee: KOREA INSTITUTE OF SCIENCE AND TECHNOLOGY, Seoul (KR)

(21) Appl. No.: 19/447,569

(22) Filed: Jan. 13, 2026

(30) Foreign Application Priority Data
Feb. 14, 2025 (KR) 10-2025-0019245

(51) Int. Cl. A61B 5/05; A61B 5/00; A61B 5/083; G16H 40/67; H04B 7/06

(52) U.S. Cl. CPC A61B 5/05; A61B 5/0833; A61B 5/7267; G16H 40/67; H04B 7/0626

(57) ABSTRACT

According to a method and device for estimating VO₂ of a user by using wireless signal around a user, user-specific characteristic data indicating characteristics of change in wireless signal around the user according to a change in a user's body is generated from channel state information collected from the wireless signal transmitted and received between a transmitter and receiver around the user, the user's VO₂ is estimated based on an output of a machine learning model by inputting the user-specific characteristic data to the machine learning model, and thus, the user may accurately estimate VO₂ without wearing a separate wearable device, such as a mask-type respiratory gas analyzer.

### CROSS-REFERENCE TO RELATED APPLICATION

This application is based on and claims priority under 35 U.S.C. § 119 to Korean Patent Application No. 10-2025-0019245, filed on Feb. 14, 2025, in the Korean Intellectual Property Office, the disclosure of which is incorporated by reference herein in its entirety.

### BACKGROUND

The present disclosure relates to a method and device for estimating VO₂ of a user.

Currently, the measurement of a person's activity or exercise amount is mainly performed by using an accelerometer, a global positioning system (GPS), and so on built in a smartphone, smartwatch, and so on. However, since the measurement may be performed on a user wearing a smartphone, smartwatch, or so on, there is inconvenience in that a device having a function for measuring a user's activity or exercise amount has to be worn. In particular, the measurement method has a problem in that the activity or exercise amount is not accurate because the measurement method measures the activity or exercise amount from a user's movement speed, movement distance, and so on.

In order to accurately measure a person's activity or exercise amount, measurement of VO₂, which means the amount of oxygen consumed by a person per minute, is required. Currently, VO₂ measurement is mostly performed by a mask-type respiratory gas analyzer. In order for a user to measure VO₂ using a respiratory gas analyzer, a device for analyzing the respiratory gas collected by a mask has to be worn in addition to a mask worn on a user's mouth, and accordingly, there is a problem that the respiratory gas analyzer may not be used during daily activity or exercise.

### SUMMARY

The present disclosure provides a method and device that may accurately estimate a user's VO₂ without wearing a separate wearable device and may quickly estimate a user's VO₂ in real time without being restricted by time and place. The present disclosure is not limited to the technical tasks described above, and other technical tasks may be derived from the following description.

According to an aspect of the present disclosure, a method of estimating VO₂ includes collecting channel state information indicating a wireless channel state between a transmitter and a receiver around a user from wireless signal transmitted and received between the transmitter and the receiver, generating user-specific characteristic data indicating characteristics of change in the wireless signal around the user according to a change in a body of the user from the collected channel state information, and estimating VO₂ of the user based on an output of a machine learning model by inputting the user-specific characteristic data to a pre-learned machine learning model.

In the generating of the user-specific characteristic data, the user-specific characteristic data indicating at least one of amplitude change characteristics and phase change characteristics of the wireless signal around the user according to a physical change of the user may be generated from the collected channel state information.

The user-specific characteristic data may be generated based on multiple amplitude values of the wireless signal around the user.

The user-specific characteristic data may be an average amplitude value and a standard deviation of the multiple amplitude values of the wireless signal around the user.

In the collecting of the channel state information, the channel state information may be collected by periodically receiving multiple Wi-Fi packets from the receiver, and each of the multiple Wi-Fi packets received from the receiver may include channel state information on the receiver.

The method of estimating VO₂ may further include extracting the multiple Wi-Fi packets including the channel state information indicating the wireless channel state between the transmitter and the receiver from the received multiple Wi-Fi packets with reference to MAC addresses of the multiple Wi-Fi packets received from the receiver.

The generating of the user-specific characteristic data may include reading multiple channel state information (CSI) values for each subcarrier from the extracted multiple Wi-Fi packets, and calculating multiple amplitude values for each subcarrier from the read multiple CSI values for each subcarrier, and the user-specific characteristic data may be generated based on the multiple amplitude values calculated for each subcarrier.

The generating of the user-specific characteristic data may further include determining packet frequencies of the extracted multiple Wi-Fi packets with reference to a timestamp value recorded in the extracted multiple Wi-Fi packets, generating an amplitude change pattern for each subcarrier by listing the multiple amplitude values calculated for each subcarrier, and interpolating the generated amplitude change pattern for each subcarrier based on the determined packet frequencies.

The generating of the user-specific characteristic data may further include calculating an average amplitude value and a standard deviation of the amplitude change pattern for each subcarrier corresponding to the interpolated result, and the user-specific characteristic data may be generated based on the average amplitude value and the standard deviation of the amplitude change pattern for each subcarrier.

The generating of the user-specific characteristic data may further include selecting one subcarrier among multiple subcarriers of each of the extracted multiple WiFi packets based on the standard deviation of the amplitude change pattern for each subcarrier, and the user-specific characteristic data may be an average amplitude value and a standard deviation of an amplitude change pattern of the selected one subcarrier.

The method of estimating VO₂ may further include generating participant-specific characteristic data indicating characteristics of change in wireless signal around a participant according to a physical change of a participant participating in learning the machine learning model from channel state information collected before the channel state information is collected, collecting a VO₂ measurement value of the participant at a timepoint when the channel state information is collected before the channel state information is collected, and training the machine learning model by using the generated participant-specific characteristic data as input data of the machine learning model and using the collected VO₂ measurement value of the participant as a label of the input data.

According to another aspect of the present disclosure, there is provided a computer-readable recording medium in which a program for causing a computer to perform the method of estimating VO₂ is recorded.

According to another aspect of the present disclosure, a VO₂ estimation device includes a collection unit configured to collect channel state information indicating a wireless channel state between a transmitter and a receiver around a user from wireless signal transmitted and received between the transmitter and the receiver, a preprocessing unit configured to generate user-specific characteristic data indicating characteristics of change in the wireless signal around the user according to a change in a body of the user from the collected channel state information, and an estimation unit configured to estimate VO₂ of the user from an output of a machine learning model by inputting the user-specific characteristic data to a pre-learned machine learning model.

### BRIEF DESCRIPTION OF THE DRAWINGS

Embodiments will be more clearly understood from the following detailed description taken in conjunction with the accompanying drawings in which:

FIG. 1 is a configuration diagram of a VO₂ estimation device according to an embodiment of the present disclosure;

FIG. 2 is a flowchart of a VO₂ estimation method according to an embodiment of the present disclosure;

FIG. 3 is a detailed flowchart of step 23 illustrated in FIG. 2;

FIG. 4 is a data structure diagram of each WiFi packet extracted in step 22 illustrated in FIG. 2;

FIG. 5 is an example diagram of a normalized amplitude change pattern in step 35 illustrated in FIG. 3; and

FIG. 6 is a flowchart of a learning process of a machine learning model used in the VO₂ estimation method illustrated in FIG. 2.

### DETAILED DESCRIPTION OF THE EMBODIMENTS

Hereinafter, embodiments of the present disclosure will be described in detail with reference to the drawings. The embodiments of the present disclosure relate to a method and system for estimating a user's oxygen intake amount by using a Wi-Fi wireless signal and for calculating an exercise index based thereon. Hereinafter, the method is briefly referred to as “an oxygen intake estimation and exercise index calculation method” and the system is briefly referred to as “an oxygen intake estimation and exercise index calculation system”.

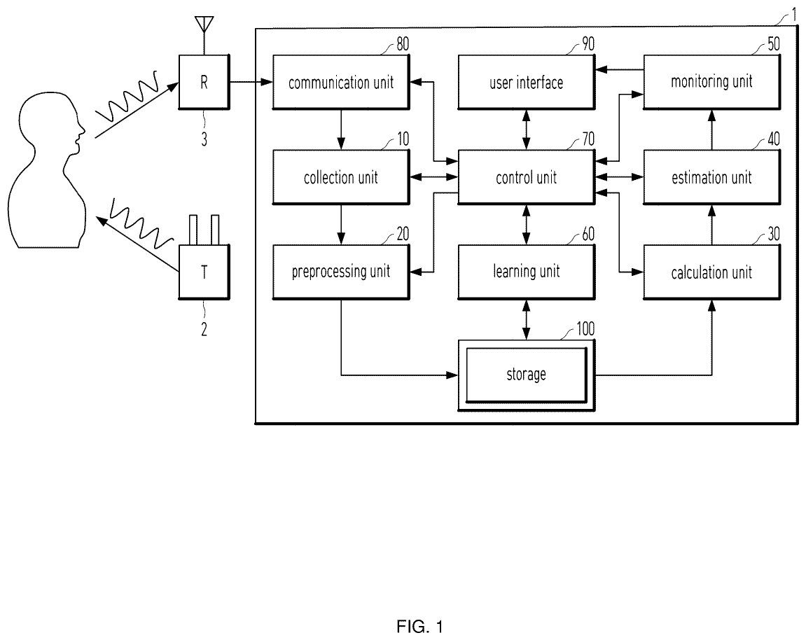

FIG. 1 is a configuration diagram of a VO₂ estimation device 1 according to an embodiment of the present disclosure. Referring to FIG. 1, the VO₂ estimation device 1 according to the present embodiment includes a collection unit 10, a preprocessing unit 20, a calculation unit 30, an estimation unit 40, a monitoring unit 50, a learning unit 60, a control unit 70, a communication unit 80, a user interface 90, and a storage 100. In order to make the present embodiment easily understandable while preventing the features of the present embodiment from being obscured, essential components of the present embodiment are illustrated in FIG. 1. Anyone having ordinary knowledge in the technical field to which the present embodiment belongs may understand that other components may be added to the configuration illustrated in FIG. 1.

The collection unit 10, the preprocessing unit 20, the calculation unit 30, the estimation unit 40, the monitoring unit 50, the learning unit 60, and the control unit 70 may be implemented by a combination of a computer processor, a recording medium, and a computer program, or may be implemented by an field programmable gate array (FPGA). The communication unit 80 performs WiFi communication with a receiver 3. The communication unit 80 may also perform communication with other electronic devices according to various communication methods. The user interface 90 may include a display panel, a touch screen, and so on. The storage 100 may include a solid state drive (SSD), a hard disk, and so on. A machine learning model may be implemented by a computer program to be stored in the storage 100. The machine learning model may also be implemented by an FPGA.

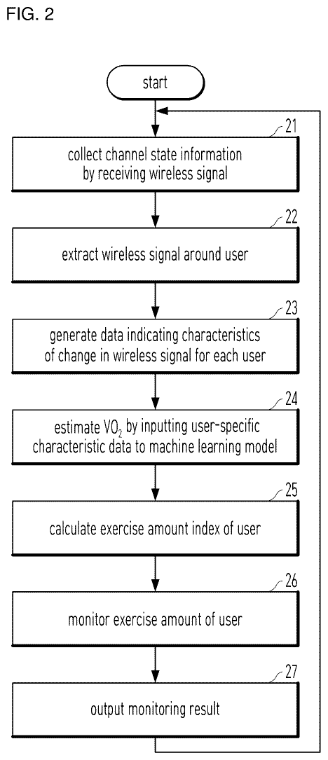

FIG. 2 is a flowchart of a VO₂ estimation method according to an embodiment of the present disclosure. Referring to FIG. 2, the VO₂ estimation method according to the present embodiment may include the following steps performed by the VO₂ estimation device 1 illustrated in FIG. 1. Hereinafter, the VO₂ estimation device 1 illustrated in FIG. 1 and the VO₂ estimation method illustrated in FIG. 2 are described in detail with reference to FIGS. 1 and 2. The control unit 70 may control operations of the collection unit 10, the preprocessing unit 20, the calculation unit 30, the estimation unit 40, the monitoring unit 50, and the learning unit 60 such that the VO₂ estimation device 1 illustrated in FIG. 1 may perform the steps illustrated in FIG. 2.

In step 21 and step 22, the collection unit 10 collects channel state information (CSI) indicating a wireless channel state between a transmitter 2 and the receiver 3 from wireless signal transmitted and received between the transmitter 2 and the receiver 3 around a user. In step 21, the collection unit 10 collects the channel state information by receiving a wireless signal including the channel state information from the receiver 3 through the communication unit 80. A representative example of the wireless signal including the channel state information may include a Wi-Fi packet. That is, the collection unit 10 collects the channel state information by periodically receiving multiple Wi-Fi packets from the receiver 3. Each of the multiple Wi-Fi packets received from the receiver 3 includes the channel state information on the receiver 3. For example, the collection unit 10 may collect 70 Wi-Fi packets per second at intervals of about 14 ms to about 18 ms.

For example, the transmitter 2 may be an access point in a Wi-Fi communication environment, and for example, the receiver 3 may be an Internet of Things (IoT) device including a low-cost chipset, such as ESP32, having a channel state information generation function. The transmitter 2 may be installed in a position in which a wireless signal transmitted therefrom may change according to a change in a user's body, such as the user's movement, and the receiver 3 may be installed in a position in which the wireless signal changed according to the change in the user's body may be received.

In step 22, the collection unit 10 may extract multiple Wi-Fi packets including channel state information indicating a wireless channel state between the transmitter 2 and the receiver 3 from the multiple Wi-Fi packets received from the receiver 3 with reference to medium access control (MAC) addresses of the multiple Wi-Fi packets received from the receiver 3. The receiver 3 may receive the Wi-Fi packets from multiple access points in addition to the transmitter 2. The collection unit 10 may extract multiple Wi-Fi packets, in which the MAC address of the transmitter 2 is recorded, from the multiple Wi-Fi packets received from the receiver 3, thereby extracting multiple Wi-Fi packets in which channel state information indicating a wireless channel state between the transmitter 2 and the receiver 3 is recorded.

In step 23, the preprocessing unit 20 may generate user-specific characteristic data indicating characteristics of change in wireless signal around a user according to a change in a user's body from the channel state information collected by the collection unit 10 in step 21 and step 22. For example, the change in the user's body may include the user's movement according to the user's exercise or the user's body deformation according to the user's heavy breathing. The preprocessing unit 20 may generate user-specific characteristic data indicating at least one of amplitude change characteristics and phase change characteristics of a wireless signal around a user according to the user's body change from the channel state information collected by the collection unit 10 in step 21 and step 22.

In the present embodiment, in order to reduce a data amount of user-specific characteristic data, the preprocessing unit 20 generates user-specific characteristic data indicating amplitude change characteristics of wireless signal around a user according to the user's physical change from the channel state information collected in step 22. Accordingly, the user-specific characteristic data is generated based on multiple amplitude values of the wireless signal around a user. For example, the user-specific characteristic data may be an average amplitude value and standard deviation of multiple amplitude values of wireless signal around a user.

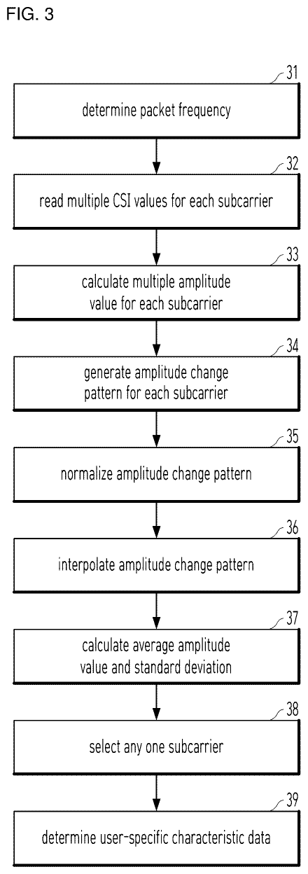

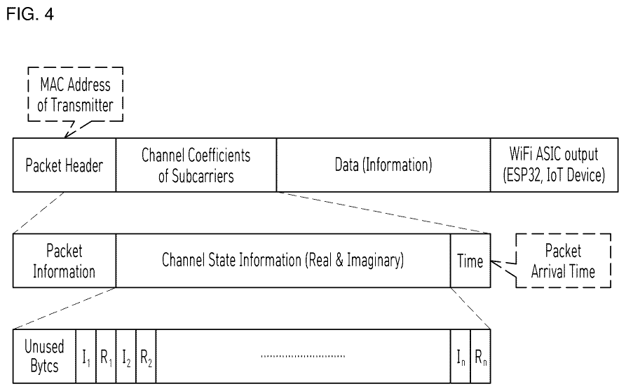

A representative example of the wireless signal around a user may include multiple Wi-Fi packets transmitted from an access point corresponding to the transmitter 2. In the present embodiment, the user-specific characteristic data may be generated using channel state information of each of multiple Wi-Fi packets transmitted from an access point. FIG. 3 is a detailed flowchart of step 23 illustrated in FIG. 2. Referring to FIG. 3, step 23 illustrated in FIG. 2 includes the following steps. FIG. 4 is a data structure diagram of each Wi-Fi packet extracted in step 22 illustrated in FIG. 2. Referring to FIG. 4, a MAC address of the transmitter 2 may be included in a header of each WiFi packet.

In step 31, the preprocessing unit 20 may determine a packet frequency of multiple WiFi packets extracted by the collection unit 10 in step 22 with reference to timestamp values recorded in the multiple WiFi packets extracted by the collection unit 10 in step 22. As illustrated in FIG. 4, the timestamp value is recorded in a time field of each WiFi packet and indicates the arrival time of each WiFi packet. The preprocessing unit 20 counts the number of WiFi packets received from the receiver 3 for one second at one-second intervals for the multiple WiFi packets extracted in step 22. The preprocessing unit 20 may perform the packet counting multiple times, for example, 10 times, and may determine a packet frequency of the multiple WiFi packets extracted in step 22 by averaging the count values.

In step 32, the preprocessing unit 20 may read multiple channel state information (CSI) values for each subcarrier from the multiple WiFi packets extracted by the collection unit 10 in step 22. Each WiFi packet may be divided into multiple subcarriers in a frequency domain by orthogonal frequency division multiplexing (OFDM) and transmitted. According to the IEEE 802.11n standard, each Wi-Fi packet may be divided into 64 subcarriers in a 20 MHz band and transmitted. Among the 64 subcarriers, CSI values are recorded in each of 52 data subcarriers. Accordingly, the multiple CSI values may be read out for each subcarrier from the multiple Wi-Fi packets extracted by the collection unit 10.

In step 33, the preprocessing unit 20 calculates multiple amplitude values for each subcarrier from the multiple CSI values read out for each subcarrier in step 32. Each CSI value is a value having a form of a complex number including a real part and an imaginary part. One amplitude value and one phase value may be derived from each CSI value. In the present embodiment, only an amplitude value is derived from each CSI value to quickly estimate a user's VO₂ in real time without being restricted by time and place using a smartphone or tablet computer having a low hardware specification. User-specific characteristic data may be generated based on multiple amplitude values derived for each subcarrier.

In step 34, the preprocessing unit 20 may list multiple amplitude values derived for each subcarrier in step 33 according to a time order of timestamp values recorded in the multiple Wi-Fi packets extracted by the collection unit 10 in step 22, and at an interval of a packet frequency determined in step 31, thereby generating an amplitude change pattern for each subcarrier. The timestamp values recorded in a certain Wi-Fi packet may become timestamp values of multiple subcarriers of the Wi-Fi packet. Accordingly, the multiple amplitude values derived for each subcarrier may have different timestamp values, that is, different packet arrival times.

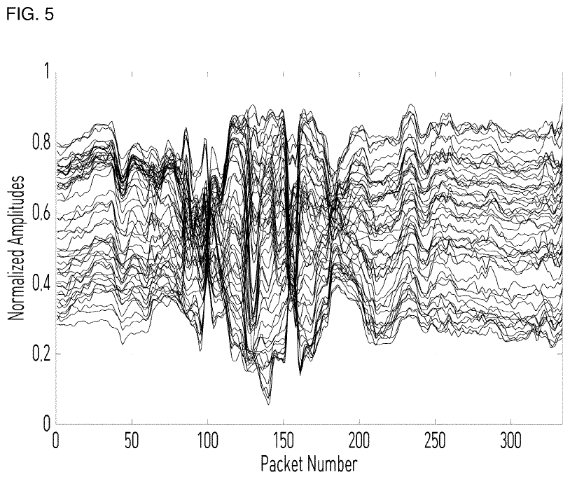

In step 35, the preprocessing unit 20 may normalize the amplitude change pattern generated for each subcarrier in step 34. The preprocessing unit 20 may normalize the amplitude change pattern in the following manner. First, the preprocessing unit 20 may remove noise from the amplitude change pattern by removing an outlier of the amplitude change pattern using a Hampel filter. Next, the preprocessing unit 20 may normalize the amplitude change pattern by adjusting each amplitude value of the amplitude change pattern using a mean shift technique. An error due to a drift phenomenon of the CSI value may be resolved by an average shift technique. FIG. 5 is an example diagram of a normalized amplitude change pattern in step 35 illustrated in FIG. 3. FIG. 5 illustrates a list of numerous amplitude values in the form of lines.

In step 36, the preprocessing unit 20 interpolates the amplitude change pattern generated for each subcarrier in step 35 based on the packet frequency determined in step 31. For example, inter-packet arrival time of multiple Wi-Fi packets extracted in step 22 may be between 2 ms and 50 ms. When there is an obstacle or so on in a movement path of the multiple Wi-Fi packets, some of the multiple Wi-Fi packets may arrive later than the other Wi-Fi packets, and some of the multiple Wi-Fi packets may be lost. Accordingly, in step 34, the amplitude change pattern for each subcarrier may be generated by listing multiple amplitude values according to intervals of packet frequencies.

In a certain amplitude change pattern generated in this way, when an interval between two adjacent amplitude values is greater than an interval between packet frequencies, this is due to packet loss. For example, when the packet frequency is 100 Hz, an interval between two adjacent amplitude values is 10 ms. In the certain amplitude change pattern, when an interval between two adjacent amplitude values is 20 ms, this means the loss of one WiFi packet, and when the interval between the two adjacent amplitude values is 30 ms, this means losses of two WiFi packets.

In this case, the preprocessing unit 20 calculates an interpolation value of two adjacent amplitude values using two adjacent amplitude values, and inserts the calculated interpolation value into the two adjacent amplitude values, thereby interpolating the amplitude change pattern generated for each subcarrier based on the packet frequency. For example, the preprocessing unit 20 may calculate an average value of two adjacent amplitude values as an interpolation value of two adjacent amplitude values when an interval between two adjacent amplitude values in the certain amplitude change pattern is 20 ms.

In step 37, the preprocessing unit 20 may calculate an average amplitude value and standard deviation of the amplitude change pattern for each subcarrier corresponding to the interpolated result in step 36. User-specific characteristic data may be generated based on the average amplitude value and standard deviation of the amplitude change pattern for each subcarrier. When the entire amplitude change pattern is input to the machine learning model, the data amount of the amplitude change pattern increases significantly, and accordingly, the calculation amount of the machine learning model may also increase significantly. Accordingly, it may be difficult to quickly estimate a user's VO₂ in real time depending on hardware performances of the VO₂ estimation device 1.

In step 38, the preprocessing unit 20 may select one subcarrier from among multiple subcarriers of each of the multiple WiFi packets extracted by the collection unit 10 in step 22 based on the standard deviation of the amplitude change pattern for each subcarrier calculated in step 37. The standard deviation of the amplitude change pattern for each subcarrier may indicate the degree of change in amplitude change pattern for each subcarrier. As a result of numerous experiments, it may be checked that the greater the degree of change in the amplitude change pattern for each subcarrier, the higher the accuracy of a user's VO₂ estimation.

In step 39, the preprocessing unit 20 determines the average amplitude value and the standard deviation of one of the subcarriers selected in step 38 as the user-specific characteristic data. In this way, the user-specific characteristic data becomes the average amplitude value and the standard deviation of one subcarrier selected in step 38. In the present embodiment, since the user-specific characteristic data input to the machine learning model becomes the average amplitude value and standard deviation of one subcarrier that may be expressed as a very small amount of data, a user's VO₂ may be estimated quickly and in real time without being restricted by time and place using a smartphone or tablet computer having a low hardware specification.

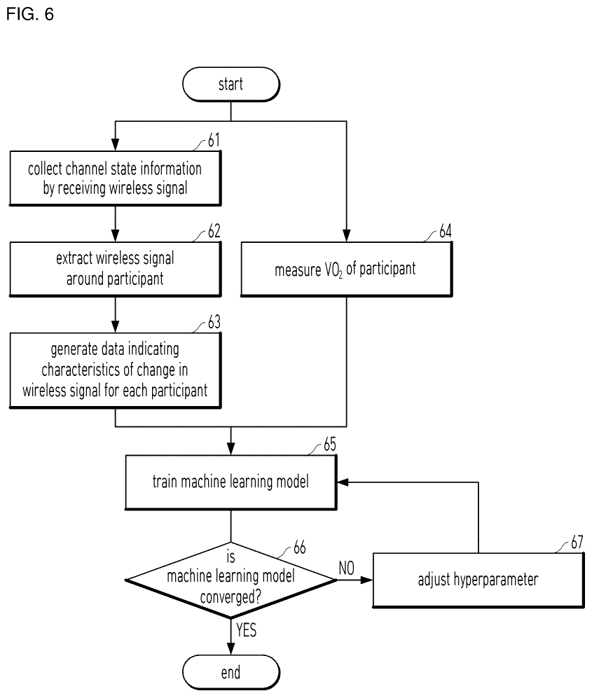

In step 24, the estimation unit 40 may estimate the user's VO₂ based on an output of the machine learning model by inputting the user-specific characteristic data generated by the preprocessing unit 20 in step 23 into the pre-learned machine learning model. Learning of the machine learning model is described in detail below with reference to FIG. 6. The machine learning models may include, for example, a linear regression model, a support vector machine models, a random forest model, a boosting model, and a deep neural network model.

In step 25, the calculation unit 30 may calculate a user's exercise volume index based on the user's VO₂ estimated by the estimation unit 40 in step 24. For example, the calculation unit 30 may calculate a metabolic equivalent of task (MET) as the user's exercise volume index based on the user's VO₂ estimated by the estimation unit 40 in step 24 according to Equation 1 below. The calculation unit 30 may also calculate the user's exercise volume index from MET according to Equation 2 below.

$$MET = \frac{{VO}_{2} ( ml / kg / min )}{3.5 ( ml / kg / min )} \qquad Equation 1$$

$$Equation 2$$

$$Calories ( kcal ) = MET \times Body Weight ( kg ) \times Exercise Time ( minutes ) \times 0.0175$$

In step 26, the monitoring unit 50 may monitor whether a user's exercise amount index calculated by the calculating unit 30 in step 25 exceeds a reference value preset by the user. For example, the monitoring unit 50 may monitor whether a user's MET calculated by the calculating unit 30 in step 25 exceeds a reference MET preset by the user. The monitoring unit 50 may monitor whether a user's calorie consumption amount calculated by the calculating unit 30 in step 25 exceeds a reference calorie consumption amount preset by the user.

In step 27, the user interface 90 may output a result monitored by the monitoring unit 50 in step 26 under the control of the control unit 70. The user interface 90 may output the result monitored by the monitoring unit 50 in step 26 in various forms, such as a text, an image, and voice. The control unit 70 may cause the user interface 90 to output the content indicating that a user's current exercise amount exceeds a reference value preset by the user when the user's exercise amount index calculated by the calculation unit 30 exceeds the reference value preset by the user. The control unit 70 may also cause the user interface 90 to output the user's exercise amount index calculated by the calculation unit 30. As step 21 to step 27 are repeated, a user's VO₂ estimation and exercise amount monitoring may be performed periodically.

FIG. 6 is a flowchart of a learning process of the machine learning model used for the VO₂ estimation method illustrated in FIG. 2. Referring to FIG. 2, the learning process of the machine learning model according to the present embodiment may include the following steps performed by the VO₂ estimation device 1 illustrated in FIG. 1. The learning of the machine learning model according to the present embodiment may be performed by a computer other than the VO₂ estimation device 1 illustrated in FIG. 1. Hereinafter, the learning process of the machine learning model according to the present embodiment will be described in detail with reference to FIG. 6.

In step 61 and step 62, the collection unit 10 may collect channel state information indicating a wireless channel state between the transmitter 2 and the receiver 3 from the wireless signal transmitted and received between the transmitter 2 and the receiver 3 around a participant participating in the learning of the machine learning model. In step 61, the collection unit 10 may collect the channel state information by receiving a wireless signal including the channel state information from the receiver 3 through the communication unit 80. In step 62, the collection unit 10 may extract multiple Wi-Fi packets including the channel state information indicating the wireless channel state between the transmitter 2 and the receiver 3 from multiple Wi-Fi packets received from the receiver 3 with reference to MAC addresses of the multiple Wi-Fi packets received from the receiver 3.

The higher the similarity between a learning environment of the machine learning model and a usage environment of the machine learning model, the higher the accuracy of a VO₂ estimation value of the machine learning model. Accordingly, it is desirable that the learning environment of the machine learning model be set as similar as possible to the usage environment of the machine learning model. For example, the transmitter 2 may be installed in a position where the wireless signal transmitted therefrom may be changed by a participant's physical change, such as the participant's movement, and the receiver 3 may be installed in a position where the wireless signal changed according to the participant's physical change may be received. In this way, the collection of the channel state information in step 61 and step 62 may be performed in the same manner as the collection of the channel state information in step 21 and step 22, and accordingly, detailed descriptions of step 61 and step 62 are replaced with the descriptions of step 21 and step 22.

In step 63, the preprocessing unit 20 may generate participant-specific characteristic data indicating characteristics of a change in wireless signal around a participant according to a change in a participant's body from the channel state information collected by the collection unit 10 in step 62. Since the learning of the machine learning model has to precede the use of the machine learning model, the channel state information collected by the collection unit 10 in step 62 may be the channel state information collected before the channel state information is collected in step 22. Since the generation of participant-specific characteristic data in step 63 may be performed in the same manner as the generation of the user-specific characteristic data in step 23, detailed description of step 63 may be replaced with the description of step 23.

In step 64, the collection unit 10 may receive the data indicating a VO₂ measurement value of a participant at the time of collecting the channel state information collected in step 61 and step 62 through the communication unit 80, thereby collecting the VO₂ measurement value of the participant at the time of collecting the channel state information collected in step 61 and step 62. VO₂ measurement of a participant may be performed in synchronization with the collection of the channel state information in step 61 and step 62 while the participant is wearing a respiratory gas analyzer. A wireless terminal connected to the respiratory gas analyzer may transmit the VO₂ measurement value of the participant to the VO₂ estimation device 1 illustrated in FIG. 1. An ergometer may also be used together for accurate VO₂ measurement.

In step 65, the learning unit 60 may train the machine learning model by using the participant-specific characteristic data generated in step 63 as input data for the machine learning model and the participant's VO₂ measurement value collected in step 64 as a label of the input data for the machine learning model. The process of inputting the participant-specific characteristic data generated in step 63 to the machine learning model and adjusting weights and so on of the machine learning model such that an output of the machine learning model approaches the participant's VO₂ measurement value may be repeated countless times. Accordingly, step 61 to step 65 may be repeated a great number of times in the learning process of the machine learning model.

In step 66, the learning unit 60 may determine whether the performance of the machine learning model learned in step 65 is converged. When the performance of the machine learning model learned in step 65 is converged as a result of the determination in step 66, the learning of the machine learning model is terminated. Otherwise, the process proceeds to step 67. A large amount of participant-specific characteristic data generated by the repetition of step 63 may be classified into learning data and test data. The learning data may be used for learning of the machine learning model in step 65, and the testing data may be used for determining whether the performance of the machine learning model is converged in step 66. Since determining whether the performance of the machine learning model is converged is the technique known to a person having common knowledge in the technical field to which the present embodiment belongs, detailed descriptions thereof are omitted.

In step 67, the learning unit 60 may adjust hyperparameters such that an output of the machine learning model approaches a VO₂ measurement value of a participant input to the machine learning model. The hyperparameters may include, for example, a learning rate, an optimization algorithm, and so on. The learning process described above may be performed for each of machine learning models, such as a linear regression model, a support vector machine, a random forest model, a boosting model, and a deep neural network, or may be performed for an ensemble model corresponding to a combination thereof. In this way, learning may be performed on several machine learning models, and a machine learning model having the highest performance may be selected from among the several machine learning models.

According to the embodiments of the present disclosure described above, user-specific characteristic data indicating characteristics of change in wireless signal around a user according to a change in a user's body may be generated from the channel state information collected from the wireless signal transmitted and received between the transmitter 2 and the receiver 3 around the user, and a user's VO₂ may be estimated based on an output of the machine learning model according to input of the user-specific characteristic data to the machine learning model, such that the user may accurately estimate the user's VO₂ without wearing a separate wearable device such as a mask-type respiratory gas analyzer.

Since the user's VO₂ may be estimated by inputting user-specific characteristic data that may be expressed with a very small amount of data to the machine learning model, for example, an average amplitude value and a standard deviation of wireless signal around the user, the user's VO₂ may be quickly estimated in real time without being restricted by time and place by using a smartphone or tablet computer having a low hardware specification. In this way, since a user's VO₂ may be estimated quickly in real time, the user may calculate an activity level or exercise level in real time without wearing a separate wearable device such as a respiratory gas analyzer.

Meanwhile, the VO₂ estimation method according to an embodiment of the present disclosure described above may be written as a program executable on a computer processor, and may be implemented by a computer that records and executes the program on a computer-readable recording medium. The computer may include all types of computers that may execute programs, such as a desktop computer, a laptop computer, a smartphone, and an embedded type computer. Also, a structure of the data used in the embodiment of the present disclosure described above may be recorded on a computer-readable recording medium through various methods and devices. The computer-readable recording medium may include storage media such as random access memory (RAM), read only memory (ROM), an SSD, magnetic storage media (for example, floppy disks, hard disks, or so on), and optical readable media (for example, compact disk (CD)-ROM, a digital video disk (DVD), and so on).

The present disclosure is described with a focus on preferred embodiments thereof. Those skilled in the art will appreciate that the present disclosure may be implemented in modified forms without departing from the essential characteristics of the present disclosure. Therefore, the disclosed embodiments should be considered from an illustrative rather than a restrictive perspective. The scope of the present disclosure is indicated by claims below, not the above description, and all differences within the equivalent scope should be interpreted as being included in the present disclosure.

The present disclosure is not limited to the effects described above, and other effects may be derived.

What is claimed is:

1. A method of estimating VO₂, the method comprising:
collecting channel state information indicating a wireless channel state between a transmitter and a receiver around a user from wireless signal transmitted and received between the transmitter and the receiver;
generating user-specific characteristic data indicating characteristics of change in the wireless signal around the user according to a change in a body of the user from the collected channel state information; and
estimating VO₂ of the user based on an output of a machine learning model by inputting the user-specific characteristic data to a pre-learned machine learning model.

2. The method of claim 1, wherein
in the generating of the user-specific characteristic data, the user-specific characteristic data indicating at least one of amplitude change characteristics and phase change characteristics of the wireless signal around the user according to a physical change of the user is generated from the collected channel state information.

3. The method of claim 2, wherein
the user-specific characteristic data is generated based on multiple amplitude values of the wireless signal around the user.

4. The method of claim 3, wherein
the user-specific characteristic data is an average amplitude value and a standard deviation of the multiple amplitude values of the wireless signal around the user.

5. The method of claim 1, wherein
in the collecting of the channel state information, the channel state information is collected by periodically receiving multiple Wi-Fi packets from the receiver, and
each of the multiple Wi-Fi packets received from the receiver includes channel state information on the receiver.

6. The method of claim 5, further comprising:
extracting the multiple Wi-Fi packets including the channel state information indicating the wireless channel state between the transmitter and the receiver from the received multiple Wi-Fi packets with reference to MAC addresses of the multiple Wi-Fi packets received from the receiver.

7. The method of claim 6, wherein
the generating of the user-specific characteristic data includes reading multiple channel state information (CSI) values for each subcarrier from the extracted multiple Wi-Fi packets, and calculating multiple amplitude values for each subcarrier from the read multiple CSI values for each subcarrier, and
the user-specific characteristic data is generated based on the multiple amplitude values calculated for each subcarrier.

8. The method of claim 7, wherein the generating of the user-specific characteristic data further includes:
determining packet frequencies of the extracted multiple Wi-Fi packets with reference to a timestamp value recorded in the extracted multiple Wi-Fi packets;
generating an amplitude change pattern for each subcarrier by listing the multiple amplitude values calculated for each subcarrier; and
interpolating the generated amplitude change pattern for each subcarrier based on the determined packet frequencies.

9. The method of claim 8, wherein
the generating of the user-specific characteristic data further includes calculating an average amplitude value and a standard deviation of the amplitude change pattern for each subcarrier corresponding to the interpolated result, and
the user-specific characteristic data is generated based on the average amplitude value and the standard deviation of the amplitude change pattern for each subcarrier.

10. The method of claim 9, wherein
the generating of the user-specific characteristic data further includes selecting one subcarrier among multiple subcarriers of each of the extracted multiple WiFi packets based on the standard deviation of the amplitude change pattern for each subcarrier, and
the user-specific characteristic data is an average amplitude value and a standard deviation of an amplitude change pattern of the selected one subcarrier.

11. The method of claim 1, further comprising:
generating participant-specific characteristic data indicating characteristics of change in wireless signal around a participant according to a physical change of a participant participating in learning the machine learning model from channel state information collected before the channel state information is collected;
collecting a VO₂ measurement value of the participant at a timepoint when the channel state information is collected before the channel state information is collected; and
training the machine learning model by using the generated participant-specific characteristic data as input data of the machine learning model and using the collected VO₂ measurement value of the participant as a label of the input data.

12. A computer-readable recording medium in which a program for causing a computer to perform the method of claim 1 is recorded.

13. A VO₂ estimation device comprising:
a collection unit configured to collect channel state information indicating a wireless channel state between a transmitter and a receiver around a user from wireless signal transmitted and received between the transmitter and the receiver;
a preprocessing unit configured to generate user-specific characteristic data indicating characteristics of change in the wireless signal around the user according to a change in a body of the user from the collected channel state information; and
an estimation unit configured to estimate VO₂ of the user from an output of a machine learning model by inputting the user-specific characteristic data to a pre-learned machine learning model.
